(19) United States
(12) Patent Application Publication
(10) Pub. No.: US 2024/0198254 A1
(43) Pub. Date: Jun. 20, 2024

(54) METHOD FOR SEPARATING A MIXTURE

(71) Applicant: K.D. PHARMA BEXBACH GMBH, Bexbach (DE)

(72) Inventors: Gideon GIESSELMANN, Tawern (DE); Roberto FRONZONI, Homburg (DE); Elia RAMOS-TERCERO, Basel (CH); Denitsa SHOPOVA-GOSPODINOVA, Schönenberg-Kübelberg (DE)

(21) Appl. No.: 18/555,587

(22) PCT Filed: Apr. 27, 2022
(86) PCT No.: PCT/EP2022/061251

§ 371 Date: Oct. 16, 2023

(30) Foreign Application Priority Data
Apr. 27, 2021 (LU) LU500093
Aug. 24, 2021 (LU) LU500567

(51) Int. Cl. B01D 15/18; C07C 37/82; C07C 51/47; C07D 311/78

(52) U.S. Cl. CPC B01D 15/185; B01D 15/1828; B01D 15/1842; C07C 37/82; C07C 51/47; C07D 311/78; B01D 2215/024

(57) ABSTRACT

A method and apparatus for isolating a first substance from a mixture by chromatography, the mixture including at least a second substance which in the chromatography is subject to a greater retention than the first substance, and having a third substance which in the chromatography is subject to a lower retention than the first substance. The mixture is supplied to a chromatography apparatus having multiple interconnected chromatography columns. The apparatus is supplied with the mixture and an eluent, and the second substance is withdrawn as an extract and the first substance as a raffinate. The third substance is withdrawn as an extract. Usefully the third substance and the second substance are withdrawn jointly as an extract.

The invention relates to a method for separating a first substance from a substance mixture by chromatography, wherein the substance mixture comprises at least one second substance which is subject to a longer retention than the first substance during chromatography, and comprises a third substance which is subject to a shorter retention than the first substance during chromatography, wherein the substance mixture is fed to an apparatus for chromatography comprising a plurality of interconnecting chromatography columns, the substance mixture and an eluent are fed to the apparatus, and the second substance is withdrawn as an extract and the first substance is withdrawn as a raffinate.

Chromatography methods of the aforementioned type are known by use. A substance mixture to be separated is passed through the chromatography column having a stationary phase comprising, for example, a so-called packing of porous material. The different substances of the substance mixture are subject to different retentions when flowing through the stationary phase, i.e. they flow through the stationary phase at different rates due to different strengths of interactions in the stationary phase. This makes separation possible. While it is possible to carry out chromatography in a so-called batch process, in which the separation always takes place in a single step, methods have been developed for carrying out chromatography continuously, in which a plurality of chromatography columns are interconnected. Such chromatography methods, which are carried out on an industrial scale, are True Moving Bed Chromatography (TMB) and Simulated Moving Bed Chromatography (SMB).

In SMB chromatography, a plurality of chromatography columns are connected in series. At different connecting points between the chromatography columns, the substance mixture and an eluent are introduced. Likewise, at other connecting points, a raffinate and an extract are withdrawn, wherein the substance or possibly substances which are subject to a shorter retention during chromatography are withdrawn as a raffinate, and the substance or substances which are subject to a longer retention during chromatography are withdrawn as an extract. Consequently, the substance mixtures can be separated by means of known chromatography methods into two different substance submixtures, depending on the operation of the chromatography, wherein a first substance submixture comprises substances which are subject to a shorter retention and a second substance submixture comprises substances which are subject to a longer retention.

The object of the invention is based on being able to carry out substance separation more flexibly by means of chromatography. This object is achieved according to the invention in that the third substance is withdrawn as an extract.

The invention makes it possible to separate a substance from the substance mixture, even if other substances are present in the substance mixture, which are subject to a longer or a shorter retention during chromatography than the substance to be separated. Whereas it was previously necessary to carry out several chromatographies in succession in order to obtain a comparable result, the substance to be separated can be separated by means of the method according to the invention by carrying out a single chromatography. The effort required to carry out the separation method is considerably reduced.

In one configuration of the invention, the chromatography columns form zones in which
a) a first zone is formed between a device for supplying the eluent and a device for discharging the extract,
b) a second zone is formed between a device for discharging the extract and a device for supplying the substance mixture,
c) a third zone is formed between a device for supplying the substance mixture and a device for discharging the raffinate, and
d) a fourth zone is formed between a device for discharging the raffinate and a device for supplying the eluent.

It is expedient to adjust the substance flows, in particular the inflows and/or outflows, via said supply and/or discharge devices in such a way that, in a zone between a device for discharging the raffinate and a device for supplying the eluent, there is such a large flow that the third substance can be withdrawn as an extract. With such a setting of the chromatography device, the third substance is forced into zone I, from which the extract is withdrawn, in contrast to that which is usual in known chromatography methods. While the third substance would be removed as a raffinate when using known chromatography methods, due to its shorter retention compared to the first substance, it is withdrawn as an extract according to the invention.

It has proven to be particularly advantageous to withdraw the third substance together with the second substance as an extract.

In one embodiment of the invention, the device for supplying the eluent, the device for discharging the extract, the device for supplying the substance mixture and the device for discharging the raffinate have a plurality of supply or discharge connections which are arranged between different chromatography columns. The supply or discharge connections are preferably provided with valves by means of which the respective substance flows, in particular the inflows and/or outflows, of the substance mixture, of the eluent, of the extract and/or of the raffinate through the respective supply or discharge connections can be adjusted.

According to the invention, the respective substance flows are adjusted in such a way that the zones in the device are alternately formed in different chromatography columns. In this way, it is possible to carry out the chromatography method according to the invention as a TMB or SMB method.

In a preferred embodiment of the invention, the content of the first substance in the raffinate is greater than the content of the first substance in the substance mixture and preferably the content of the second and/or the third substance in the extract is greater than the content of the second or the third substance in the substance mixture. It is expedient to maximize the content of the first substance in the raffinate and in particular to maximize the content of the second and/or the third substance in the extract.

It is expedient that the content of the first substance in the extract is less than the content of the first substance in the substance mixture and preferably the content of the second and/or the third substance in the raffinate is less than the content of the second or third substance in the substance mixture.

Preferably, the content of the first substance in the extract is less than the content of the first substance in the raffinate and preferably the content of the second and/or third substance in the raffinate is greater than the content of the second or third substance in the extract.

In a particularly preferred embodiment of the invention, the respective substance flows are adjusted in such a way that at least one of the aforementioned content ratios is achieved in the raffinate or extract. Preferably, the adjustment is made continuously, particularly preferably as a regulation and/or control system.

The chromatography apparatus expediently comprises at least one device for determining a substance property of the raffinate and/or of the extract, and optionally also for determining a substance property of the substance mixture and/or of the eluent, in particular the content of an ingredient of the raffinate, of the extract, of the substance mixture and/or of the eluent being determined.

It is expedient that the flows of the substance mixture, of the eluent, of the raffinate and/or of the extract in the device are adjusted, particularly preferably controlled, as a function of a result of a determination of the substance property by means of the sensor.

In one configuration of the invention, it is controlled and/or regulated in such a way that the content of the first substance in the raffinate is maximized and preferably the content of the second and/or the third substance in the raffinate is minimized.

In one embodiment of the invention, the substance mixture is or comprises a fatty acid mixture, preferably a mixture of unsaturated, in particular polyunsaturated fatty acids. Particularly preferably, the fatty acid mixture comprises omega-3 fatty acids.

The first substance to be separated from the fatty acid mixture is expediently a polyunsaturated fatty acid, preferably an omega-3 fatty acid, particularly preferably eicosapentaenoic acid (EPA) and/or docosahexaenoic acid (DHA). If EPA is separated as the first substance from the substance mixture, DHA and/or arachidonic acid (ARA) may form the second substance, which is subject to longer retention during chromatography. The third substance, which is subject to longer retention during chromatography than EPA and is withdrawn as an extract, may be stearidonic acid (SDA). The aim of withdrawing the omega-3 fatty acid, in particular eicosapentaenoic acid (EPA) and/or docosahexaenoic acid (DHA), can be to produce a product that comprises the respective omega-3 fatty acid at a particularly high concentration.

The method could also be carried out with the aim of reducing the content of a particular substance in the substance mixture. For example, by using the method, arachidonic acid could be withdrawn as the first substance and thus as a raffinate from an omega-3 fatty acid in order to reduce the content of arachidonic acid. The target product would then be withdrawn as an extract.

In a further embodiment of the invention, the substance mixture is or comprises a carboxylic acid mixture, preferably a cannabinoid-containing mixture. Expediently, the first substance is cannabidiol (CBD). Alternatively, tetrahydrocannabinol (THC), in particular Δ9-THC and/or Δ8-THC, could form the first substance. This proves to be advantageous when THC is to be removed from a carboxylic acid mixture, in particular from a cannabinoid.

If CBD is separated from the carboxylic acid as the first substance, cannabidivarin (CBDV), tetrahydrocannabivarin (THCV) and/or cannabidivarinic acid (CBDVA) may form in the second substance. The third substance may be cannabigerol (CBG), tetrahydrocannabibutolic acid (THCBA), Δ9- and/or Δ8-THC, and/or cannibigerolic acid (CBGA).

In a further configuration of the invention, the substance mixture is or comprises a mixture of pre-resolving mediators (PRM), preferably 18-HEPE, 17-HDHA and/or 14-HDHA, and/or specialized pre-resolving mediators (SPM), preferably lipoxins, resolvins, protectins and/or maresins.

In a further embodiment of the invention, the first substance is a metabolite of a polyunsaturated fatty acid, preferably of eicosapentaenoic acid (EPA) and/or of docosahexaenoic acid (DHA) and/or of docosapentaenoic acid (DPA) or has the same chemical composition as the metabolite.

In one embodiment of the invention, the method is expediently carried out such that at least one pre-resolving mediator (PRM) and/or a specialized pre-resolving mediator (SPM), which is or are preferably derived from EPA, DHA or/and DPA, is/are separated from the substance mixture.

The pre-resolving mediator (PRM) is preferably at least one substance from the group of substances comprising 18-HEPE, 17-HDHA, 14-HDHA.

The specialized pre-resolving mediator (SPM) is preferably at least one substance from the group of substances comprising lipoxin, resolvin, protectin, maresin.

The lipoxin is preferably at least one substance selected from the group of substances comprising LxA4 (5S,6R,15S-trihydroxy-7E,9E,11Z,13E-ETE), LxB4 (5S,14R,15S-trihydroxy-6E,8Z,10E,12E-ETE), 15-epi-LxA4 (5S,6R,15R-trihydroxy-7E,9E,11Z,13E-eicosatetraenoic acid), 15-epi-LxB4 (5S,14R,15R-trihydroxy-6E,8Z,10E,12E-eicosatrienoic acid).

The resolvin is advantageously derived from EPA, DHA or/and from DPA.

The resolvin is preferably at least
one substance from the group of substances comprising RvE1 (5S,12R,18R-trihydroxy-6Z,8E,10E,14Z,16E-EPA), 18S-RvE1 (5S,12R,18S-trihydroxy-6Z,8E,10E,14Z,16E-EPA), RvE2 (5S,18R-dihydroxy-6E,8Z,11Z,14Z,16E-EPA), RvE3 (17R,18R/S-dihydroxy-5Z,8Z,11Z,13E,15E-EPA), and/or
one substance from the group of substances comprising RvD1 (7S,8R,17S-trihydroxy-4Z,9E,11E,13Z,15E,19Z-DHA), RvD2 (7S,16R,17S-trihydroxy-4Z,8E,10Z,12E,14E,19Z-DHA), RvD3 (4S,11R,17S-trihydroxy-5Z,7E,9E,13Z,15E,19Z-DHA), RvD4 (4S,5R,17S-trihydroxy-6E,8E,10Z,13Z,15E,19Z-DHA), RvD5 (7S,17S-dihydroxy-4Z,8E,10Z,13Z,15E,19Z-DHA), RvD6 (4S,17S-dihydroxy-5E,7Z,10Z,13Z,15E,19Z-DHA), and/or
one substance from the group of substances comprising RvT1 (7,13R,20-trihydroxy-8E,10Z,14E,16Z,18E-DPA), RvT2 (7,8,13R-trihydroxy-9E,11E,14E,16Z,19Z-DPA), RvT3 (7,12,13R-trihydroxy-8Z,10E,14E,16Z,19Z-DPA), RvT4 (7,13R-dihydroxy-8E,10Z,14E,16Z,19Z-DPA).

The protectin is expediently derived from DHA and/or from DPA. The protectin is preferably at least
one substance from the group of substances comprising PD1 or NPD1 (10R,17S-dihydroxy-4Z,7Z,11E,13E,15Z,19Z-DHA), PDX (10S,17S-dihydroxy-4Z,7Z,11E,13Z,15E,19Z-DHA), 22-hydroxy-PD1 (10R,17S,22-trihydroxy-4Z,7Z,11E,13E,15Z,19Z-DHA), 17-epi-PD1 or AT-PD1 (10R,17R-dihydroxy-4Z,7Z,11E,13E,15Z,19Z-DHA), 10-epi-PD1 or ent-AT-NPD1 (10S,17S-dihydroxy-4Z,7Z,11E,13E,15Z,19Z-DHA) and/or
one substance from the group of substances comprising PD1n-3 (10,17-dihydroxy-7,11,13,15,19-DPA), PD2n-3 (16,17-dihydroxy-7,10,12,14,19-DPA).

The maresin is expediently derived from DHA or/and from DPA. The maresin is preferably at least
one substance from the group of substances comprising MaR1 (7R,14S-dihydroxy-4Z,8E,10E,12Z,16Z,19Z-DHA), MaR2 (13R,14S-dihydroxy-4Z,7Z,9E,11E,16Z,19Z-DHA), 7-epi-MaR1 (7S,14S-dihydroxy-4Z,8E,10Z,12E,16Z,19Z-DHA), MaR-L1 (14S,22-dihydroxy-4Z,7Z,10Z,12E,16Z,19Z-DHA), MaR-L2 (14R,22-dihydroxy-4Z,7Z,10Z,12E,16Z,19Z-DHA), and/or
one substance from the group of substances comprising MaR1n-3 (7S,14S-dihydroxy-8E,10E,12Z,16Z,19Z-DPA), MaR2n-3 (13,14-dihydroxy-7,9,11,16,19-DPA), MaR3n-3 (13,14-dihydroxy-7,9,11,16,19-DPA).

The invention also relates to an apparatus with which the separation method described can be carried out. In addition to said chromatographic columns, said supply and/or discharge devices, the apparatus preferably comprises a device for determining a substance property of the raffinate and/or of the extract, in particular for determining the content of an ingredient. The detection device is preferably used to determine physical properties, in particular light absorption, fluorescence, light scattering and/or thermal conductivity, and/or chemical properties, for example coloring or the like, and thereby to determine the substance content.

The apparatus preferably further comprises a device for adjusting a substance flow, in particular an inflow and/or outflow, of the substance mixture, of the eluent, of the raffinate and/or of the extract.

In a particularly preferred embodiment of the invention, the apparatus has a device for regulating and/or controlling substance flows, in particular inflows and/or outflows, via the supply and/or discharge devices, as a function of a result of the determination by means of the detection device. Preferably, the regulation and/or control device is provided to adjust the respective substance flows such that at least one of the aforementioned content ratios in the raffinate or extract is achieved.

The invention further relates to a computer program product by means of which the method according to the invention can be carried out. The computer program product can expediently be loaded directly into the internal memory of a digital computer and comprises software sections with which, when the computer program is running on a computer, steps of the method according to the invention can be carried out. The computer program product preferably has interfaces for interacting with an apparatus according to the invention. The computer program product may form or be part of said control and/or regulating device. Preferably, the computer program product, in particular due to the interfaces, is provided for the purpose of processing the result of the determination by means of the detection device and adjusting the supply and/or discharge devices.

Said computer program product is expediently designed to perform one, more or all of the process steps when running on the computer.

The computer program is preferably a computer program product stored on a volatile or non-volatile data carrier, preferably RAM, ROM, DRAM, SRAM, EPROM, EEPROM or the like, or a device, in particular a personal computer, a device with an embedded processor, or a signal sequence representing data suitable for transmission via a computer network, in particular the internet. The invention further relates to a data carrier signal which transmits said computer product.

The invention is elucidated in more detail below with reference to working examples and the accompanying figures which relate to the working examples. They show:

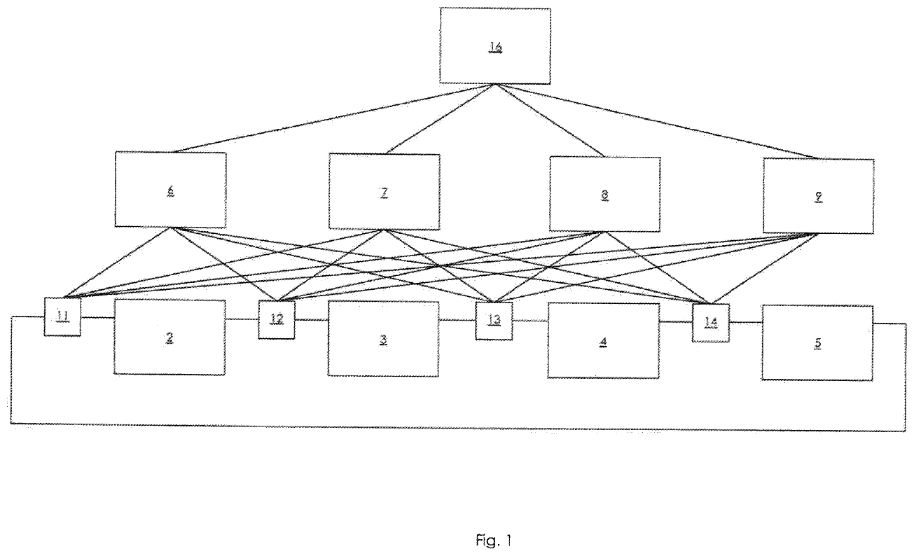

FIG. 1 schematic of an apparatus according to the invention,

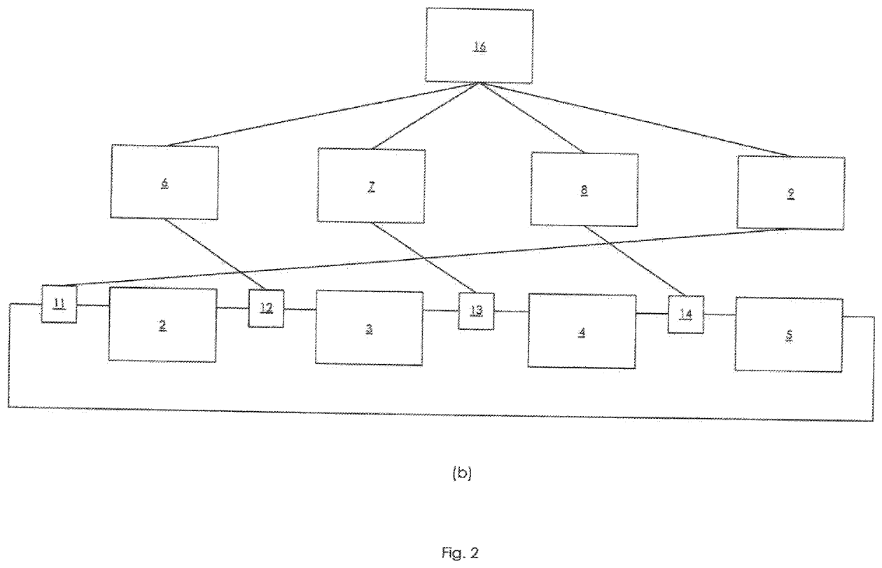

FIG. 2 the apparatus according to FIG. 1 in different positions and

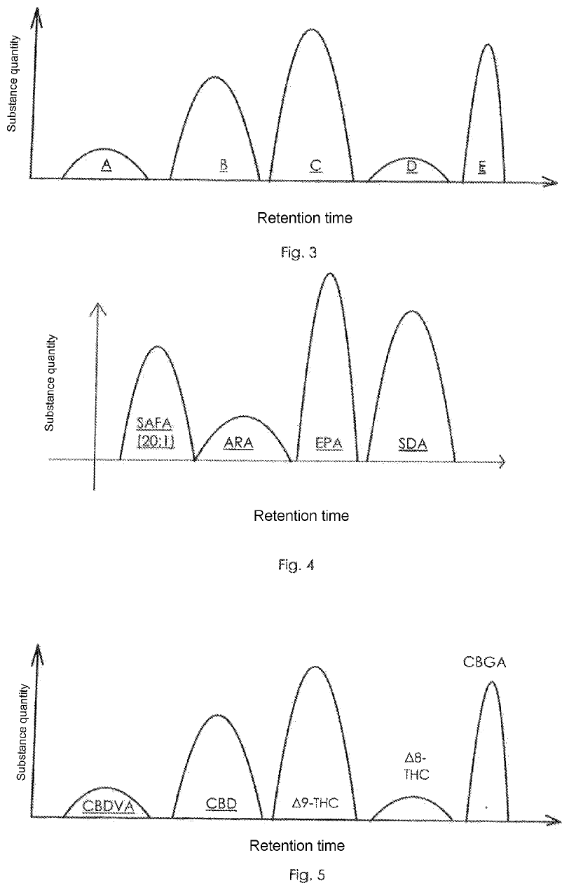

FIG. 3 a schematic graph of retention times and

FIGS. 4 and 5 schematic graphs with retention times of substances of different substance mixtures.

FIG. 1 is a schematic representation of a chromatography apparatus 1 according to the invention, comprising chromatography columns 2,3,4,5, which are connected to each other in such a way that they are in flow communication with each other. Connections 10,11,12,13 are arranged between the chromatography columns. Each of the connections 10,11,12,13 is connected to a device 6 for supplying an eluent, a device 7 for discharging an extract, a device 8 for supplying a substance mixture and a device 9 for discharging a raffinate. Said devices 6,7,8,9 each have valves by means of which substance flows, in particular inflows and/or outflows of the eluent, of the extract, of the substance mixture or of the raffinate can be adjusted, in particular the extent of the respective flows.

The apparatus comprises a sensor 14 for determining the content of a first substance in the extract and a sensor 15 for determining the content of the first substance in the raffinate. In addition, the apparatus 1 has a device 16 for regulating and/or controlling said inflows and/or outflows, in particular by adjusting said valves. The regulation and/or control device 16 preferably comprises an embedded processor on which a computer program is stored, which is provided to carry out the regulation and/or control by processing data provided by the sensors 14, 15. Depending on the open or closed position of the various valves, the positions at which the eluent or the substance mixture are supplied and the extract or the raffinate are discharged can be set. Accordingly, zones I,II,III,IV can be alternately formed in the various chromatography columns 2,3,4,5, wherein
a) a first zone I is respectively formed between a connection via which the eluent is supplied and a connection via which the extract is discharged,
b) a second zone II is formed between a connection via which the extract is discharged, and a connection via which the substance mixture is supplied,
c) a third zone III is formed between a connection via which the substance mixture is supplied and a connection via which the raffinate is discharged, and
d) a fourth zone IV is formed between a connection via which the raffinate is discharged and a connection via which the eluent is supplied.

The control and/or regulation device 16 is configured to adjust positions of the valves, depending on signals from the sensors 16, in order to adjust substance flows, in particular inflows and/or outflows. By adjusting the various valves, the zones I,II,III,IV can be assigned differently in the chromatography columns 2,3,4,5. The adjustment of the valve positions is done in such a way that the first substance can be withdrawn as the raffinate. FIG. 2a shows the apparatus 1 according to the invention in a valve switch position, in which chromatography column 2 forms zone I, chromatography column 3 forms zone II, chromatography column 4 forms zone III and chromatography column 5 forms zone IV.

By switching over the valves, zones I,II,III,IV can be formed respectively in different chromatography columns 2,3,4,5, for example, with appropriate valve switching, as shown in FIG. 2b, chromatography column 2 can form zone IV, chromatography column 3 can form zone I, chromatography column 4 can form zone II and chromatography column 5 can form zone III. The valve switching is carried out essentially as known from the known TMB or SMB chromatography methods, but taking into account the conditions elucidated below.

Implementation of the method according to the invention is elucidated in more detail with reference to FIG. 3, which shows retention times of different substances A,B,C,D and E of a substance mixture. Substance D is to be separated out from the substance mixture. As shown in FIG. 3, substance D has a longer retention time than substances A,B,C and a shorter retention time than substance E. In order to be able to withdraw substance D as raffinate, the inflows and outflows are adjusted in such a way that substance D can be withdrawn as raffinate after flowing through zone III. The substances A,B,C, which have a shorter retention time than substance D, are withdrawn after zone I as extract. In accordance with the invention, the inflows and outflows are furthermore adjusted in particular in such a way that such a flow is formed in zone IV that substance E is forced into zone I and can be withdrawn together with substances A,B,C in zone I as extract.

In a first specific working example, the separation method according to the invention is carried out on a substance mixture of polyunsaturated fatty acids. The substance mixture, in particular an oil, for example a fish oil, an algae oil or/and a linseed oil, comprises DHA, ARA, EPA and SDA. The corresponding retention times are shown in FIG. 4. EPA is to be withdrawn as raffinate. DHA and ARA have shorter retention times than EPA. SDA, on the other hand, has a longer retention time. While it would be necessary according to conventional chromatography methods to first carry out a substance separation in accordance with DHA and ARA on the one hand and EPA and SDA on the other hand and then to carry out a further separation of the already separated part comprising EPA and SDA in order to separate the two components,

TABLE 1

| | | | | | | | | Raffinate | | |
|---|---|---|---|---|---|---|---|---|---|---|
| | | | | | | | | contents | | |
| Q_{I} | Q_{II} | Q_{III} | Q_{IV} | Q_{Eluent} | Q_{SM} | Q_{Raf} | Q_{Extr} | % | % | % |
| mL/min | mL/min | mL/min | mL/min | mL/min | mL/min | mL/min | mL/min | SDA | EPA | DHA |
| 353 | 263.4 | 265 | 190 | 163 | 1.6 | 75 | 89.6 | 4.10% | 91.7% | 0.0% |
| 353 | 263.4 | 265 | 200 | 153 | 1.6 | 65 | 89.6 | 4.40% | 90.7% | 0.0% |
| 353 | 263.4 | 265 | 210 | 143 | 1.6 | 55 | 89.6 | 4.50% | 91.2% | 0.0% |
| 353 | 263.4 | 265 | 220 | 133 | 1.6 | 45 | 89.6 | 3.70% | 93.2% | 0.0% |
| 353 | 263.4 | 265 | 230 | 123 | 1.6 | 35 | 89.6 | 3.00% | 93.0% | 0.0% |

when carrying out the method according to the invention, as elucidated above, not only are DHA and ARA withdrawn as extract, but also SDA is withdrawn as extract if the inflows and outflows are adjusted accordingly. The EPA is withdrawn exclusively as raffinate.

Table 1 shows that different contents of SDA and EPA can be obtained in the raffinate if different flows Q_{IV} are set in zone IV. The lowest content of SDA can be obtained when a comparatively large flow is set in zone IV. The settings of the different flows in the zones I,II,III,IV are achieved, as can also be seen from Table 1, by setting different substance flows, in particular inflows and/or outflows, of the eluent (Q_{Eluent}), of the substance mixture (Q_{SM}), of the raffinate (Q_{Raf}) and of the extract (Q_{Extr}).

In a further working example, it is intended to withdraw ARA as a raffinate from the same substance mixture for which the retention times are shown in FIG. 4, in order to free the substance mixture from the arachidonic acid. In this case, the substance flows are adjusted in such a way that the EPA and SDA are forced into zone I so that they can be withdrawn together with the DHA as an extract. It is understood that for this purpose larger flows have to be provided in zone IV compared to the conventional application of SMB chromatography.

In a further working example, the separation method is carried out on a substance mixture comprising a cannabidiol. FIG. 5 shows a schematic diagram of the retention times of individual selected substances of the substance mixture, namely those of CBDVA, CBD, Δ9-THC, Δ8-THC and CBGA.

The intention is to obtain CBD from the substance mixture, for which purpose the substance flows are adjusted when carrying out the chromatography in such a way that CBD can be withdrawn as the only raffinate. CBDVA, which has a shorter retention time than CBD, and Δ9-THC, Δ8-THC and CBGA, which have a longer retention time than CBD, are withdrawn together as an extract.

Furthermore, by means of the method according to the invention, it is possible to free the latter cannabidiol substance mixture from THC by adjusting the substance flows in such a way that Δ9-THC and Δ8-THC are withdrawn together as raffinate. CBDVA and CBD, which have a shorter retention time than Δ9-THC and Δ8-THC, and CBGA, which has a longer retention time than Δ9-THC and Δ8-THC, are withdrawn together as an extract.

1-19. (canceled)

20. A method for separating a first substance from a substance mixture by chromatography, wherein the substance mixture comprises the first substance, at least one second substance and at least one third substance, the method comprising the steps of: subjecting the second substance to a longer retention than the first substance during chromatography; subjecting the least one third substance which is subject to a shorter retention than the first substance during chromatography; feeding the substance mixture and an eluent to an apparatus for chromatography comprising a plurality of interconnected chromatography columns; withdrawing the second substance as an extract and the first substance as a raffinate; and withdrawing the third substance as an extract.

21. The method according to claim 20, including withdrawing the third substance and the second substance jointly as an extract.

22. The method according to claim 20, including forming zones in the chromatography columns, the zones including
a) a first zone formed between a device for supplying the eluent and a device for discharging the extract,
b) a second zone formed between the device for discharging the extract and a device for supplying the substance mixture,
c) a third zone formed between the device for supplying the substance mixture and a device for discharging the raffinate, and
d) a fourth zone formed between the device for discharging the raffinate and the device for supplying the eluent.

23. The method according to claim 22, including adjusting substance flows via the supply and/or discharge devices so that in the fourth zone between the device for discharging the raffinate and the device for supplying the eluent, there is such a large flow that the third substance is removed as an extract.

24. The method according to claim 20, wherein a content of the first substance in the raffinate is greater than a content of the first substance in the substance mixture.

25. The method according to claim 24, wherein a content of the second and/or the third substance in the extract is greater than a content of the second or the third substance in the substance mixture.

26. The method according to claim 22, wherein the device for supplying the eluent, the device for discharging the extract, the device for supplying the substance mixture and the device for discharging the raffinate comprise a plurality of supply and discharge connections between different chromatography columns, the method including adjusting respective substance flows through the supply and discharge connections so that the zones are formed alternately in different chromatography columns.

27. The method according to claim 20, including determining a substance property of the raffinate and/or of the extract using at least one sensor, and adjusting flows of the substance mixture, of the eluent, of the raffinate and/or of the extract in the apparatus as a function of a result of the determination of the substance property.

28. The method according to claim 27, wherein the substance property is a content of an ingredient.

29. The method according to claim 20, wherein the substance mixture is or comprises a fatty acid mixture and/or a carboxylic acid mixture and/or a mixture of pre-resolving mediators and/or of specialized pre-resolving mediators.

30. The method according to claim 29, wherein the fatty acid mixture is a mixture of unsaturated fatty acids.

31. The method according to claim 30, wherein the fatty acid mixture is a mixture of polyunsaturated fatty acids.

32. The method according to claim 29, wherein the carboxylic acid mixture is a mixture of cannabinoids.

33. The method according to claim 29, wherein the mixture of pre-resolving mediators includes 18-HEPE, 17-HDHA and/or 14-HDHA.

34. The method according to claim 29, wherein the mixture of specialized pre-resolving mediators includes lipoxins, resolvins, protectins and/or maresins.

35. The method according to claim 20, wherein the first substance is a polyunsaturated fatty acid or cannabidiol or tetrahydrocannabinol.

36. The method according to claim 35, wherein the first substance is eicosapentaenoic acid and/or docosahexaenoic acid.

37. The method according to claim 20, wherein
a) the third substance is stearidonic acid and/or the second substance is arachidonic acid and/or docosahexaenoic acid,
b) the third substance is stearidonic acid and/or eicosapentaenoic acid and/or the second substance is arachidonic acid,
or
c) the third substance is tetrahydrocannabinol, CBGA and/or CBG and/or the second substance is CBDVA, THCV and/or CBDV.

38. The method according to claim 20, wherein the first substance is a metabolite of a polyunsaturated fatty acid or has the same composition as the metabolite.

39. The method according to claim 38, wherein the first substance is a metabolite of eicosapentaenoic acid and/or docosahexaenoic acid (DHA) and/or docosapentaenoic acid.

40. The method according to claim 20, including withdrawing at least one pre-resolving mediator, preferably 18-HEPE, 17-HDHA and/or 14-HDHA, and/or at least one specialized pre-resolving mediator, preferably lipoxins, resolvins, protectins and/or maresins, is withdrawn from the substance mixture.

41. The method according to claim 40, including withdrawing 18-HDPE, 17-HDHA and/or 14-HDHA from the substance mixture.

42. The method according to claim 40, including withdrawing lipoxins, resolvins, protectins and/or maresins from the substance mixture.

43. The method according to claim 20, wherein the first substance is a pre-resolving mediator and/or a specialized pre-resolving mediator.

44. The method according to claim 20, wherein,
a) the third substance is a pre-resolving mediator and/or a specialized pre-resolving mediator and/or the second substance is arachidonic acid and/or docosahexaenoic acid,
b) the third substance is a pre-resolving mediator and/or a specialized pre-resolving mediator and/or the second substance is eicosapentaenoic acid and/or arachidonic acid
or
c) the third substance is a pre-resolving mediator and/or a specialized pre-resolving mediator and/or the second substance is docosapentaenoic acid.

45. An apparatus for separating a first substance from a substance mixture by chromatography, comprising: a plurality of interconnected chromatography columns; a device for supplying the substance mixture; a device for supplying an eluent; a device for discharging an extract; and a device for discharging a raffinate, wherein the substance mixture comprises at least one second substance that is subject to a longer retention than the first substance during chromatography, and comprises at least one third substance that is subject to a shorter retention than the first substance during chromatography, wherein the apparatus is configured to withdraw the second substance as an extract and the first substance as a raffinate, wherein the apparatus is further configured to remove the third substance as an extract.

46. The apparatus according to claim 45, further comprising a device for adjusting an inflow and/or outflow of the substance mixture, of the eluent, of the raffinate and/or of the extract so that the third substance is withdrawn together with the second substance as an extract.

47. The apparatus according to claim 45, further comprising at least one sensor for determining a substance property of the raffinate and/or of the extract, and a device for regulating and/or controlling substance flows via the supply and/or discharge devices as a function of a result of the determination by the at least one sensor.

48. A computer program product that is loadable directly into an internal memory of a digital computer, the computer program product comprising software sections that, when the computer program product is running on a computer, carry out steps of a method for controlling and/or regulating a substance separation apparatus, wherein the substance separation apparatus comprises a plurality of interconnected chromatography columns and is provided for separating a first substance from a substance mixture, wherein the substance mixture comprises at least one second substance that is subject to a longer retention than the first substance when chromatography is carried out, and comprises a third substance that is subject to a shorter retention than the first substance during chromatography, the substance mixture and an eluent being fed to the apparatus, and the second substance being withdrawn as an extract and the first substance being withdrawn as a raffinate, wherein substance flows of the substance mixture, of the eluent, of the raffinate and/or of the extract in the apparatus are adjusted so that the third substance is withdrawn as an extract.

49. A data carrier signal that transmits the computer program product according to claim 48.
